(19) United States
(12) Patent Application Publication
(10) Pub. No.: US 2024/0390664 A1
(43) Pub. Date: Nov. 28, 2024

(54) LUER FITTING DISINFECTION DEVICE

(71) Applicant: Becton, Dickinson and Company, Franklin Lakes, NJ (US)

(72) Inventors: Manish Kumar, Bengaluru (IN); Shishir Prasad, Ramsey, NJ (US); Praveen Nalawade, Belgaum (IN); Karthik MR, Bangalore (IN)

(73) Assignee: Becton, Dickinson and Company, Franklin Lakes, NJ (US)

(21) Appl. No.: 18/202,599

(22) Filed: May 26, 2023

(51) Int. Cl. A61M 39/16

(52) U.S. Cl. CPC A61M 39/16

(57) ABSTRACT

A female Luer fitting disinfection device comprises a container comprising a bottom surface, a side wall, and an open end; a porous foam insert disposed within the container, the porous foam insert comprising a central region. A plug is positioned within the central region, the plug sized and shaped to fit within a lumen of the female Luer fitting, and a liquid disinfectant is in the container and absorbed within the porous foam insert. A sealing web covering the open end of the container. A method of disinfecting a female Luer fitting is also provided.

### TECHNICAL FIELD

The present disclosure generally relates to a disinfection device configured to disinfect medical devices, for example, injection hubs and access ports having a Luer fitting, for example, a female Luer fitting.

### BACKGROUND

Vascular access devices (VADs) are commonly used therapeutic devices and include intravenous (IV) catheters, injection hubs and access ports having a Luer fitting. Bacteria and other microorganisms can enter a patient's vascular system from access hubs and ports/valves upon connection to the VAD to deliver the fluid or pharmaceutical. Each access hub (or port/valve or connection) is associated with some risk of transmitting a catheter related bloodstream infection (CRBSI), which can be costly and potentially lethal.

To decrease CRBSI cases and to ensure VADs are used and maintained correctly, standards of practice have been developed, which include disinfecting and cleaning procedures. Disinfection devices have been added to the Society for Healthcare Epidemiology of America (SHEA) guidelines. Disinfection devices may also be incorporated into the Infusion Nurses Standards (INS) guidelines.

In developed markets, when utilizing an IV catheter, a needleless connector will typically be used to close off the system and then subsequently accessed to administer medication or other necessary fluids via the catheter to the patient. Infusion Nurses Standards (INS) Standards of Practice recommend the use of a needleless connector and state that the connector should be disinfected. The disinfection of the needleless connector is intended to aid in the reduction of bacteria that could be living on the surface of the medical device and possibly lead to a variety of catheter related complications including CRBSI. Nurses will typically utilize a 70% isopropyl alcohol (IPA) swab to complete this disinfection task by doing what is known as “scrubbing the hub.” However, compliance to this practice varies from user to user, thus resulting in inconsistent disinfection practices and results depending on the user. In addition to a lack of compliance to scrubbing the hub, clinicians have observed that there is often a variation in scrub time, dry time and the number of times the needleless connector is scrubbed. However, disinfection devices currently available fail to contact the entire face and sides of an injection port at all times during the swabbing.

In addition, over the past two decades, a growing number of studies have highlighted concern over the risk of intraluminal contamination from open female Luer fittings (stopcocks, catheter hubs, etc.). Surveys of clinicians such as nurses indicate beliefs by clinicians that 12% that disinfection of a stopcock is not possible because it is an open lumen.

Stopcock lumens have been observed to be left open in practice, resulting contaminated stopcocks, which leads to contamination of central venous catheter tips and contamination of parenteral fluid due to retrograde flow. The aforementioned commonly used disinfection devices, e.g., alcohol pads cannot disinfect the internal portion of lumens where bacterial colonization is generally present. While disinfecting caps for open female Luer fittings are available, they cannot scrub and disinfect the internal lumen of open female Luers when these lumens are accessed by multiple devices (e.g., flush, lock, medication syringes and male Luer ends of IV sets). There is a need for a disinfection device configured to actively disinfect open female Luer fittings.

### SUMMARY

In a first embodiment, a female Luer fitting disinfection device comprises a container comprising a bottom surface, a side wall, and an open end; a porous foam insert disposed within the container, the porous foam insert comprising a central region; a plug positioned within the central region, the plug sized and shaped to fit within a lumen of the female Luer fitting; a liquid disinfectant in the container and absorbed within the porous foam insert; and a sealing web covering the open end of the container.

In a second embodiment, a method of disinfecting a female Luer fitting comprises inserting the female Luer fitting having a lumen into a container including a porous foam insert; sealing the lumen with a plug located in the container to prevent fluid ingress into the lumen; and pressing the female Luer fitting against the porous foam insert and rotating the container with respect to the female Luer fitting.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only typical embodiments of this disclosure and are therefore not to be considered limiting of its scope, for the disclosure may admit to other equally effective embodiments.

FIG. 1 is an isometric view of a medical device including a Luer fitting according to an embodiment of the disclosure;

FIG. 2 is an exploded side view of a Luer fitting disinfection device according to an embodiment of the disclosure;

FIG. 3 is a cross-sectional view of the female Luer fitting disinfection device shown in FIG. 2;

FIG. 4 is a cross-sectional view of the female Luer fitting disinfection device shown in FIG. 3 in an assembled and ready-to-use configuration;

FIG. 5 is a perspective view of the female Luer fitting disinfection device shown in FIG. 4;

FIG. 6 is a top perspective view of the sealing web of the female Luer fitting disinfection device shown in FIG. 4;

FIG. 7 is a side view of the plug of the female Luer fitting disinfection device shown in FIG. 4;

FIG. 8 is a top perspective view of the porous foam insert of the female Luer fitting disinfection device shown in FIG. 4;

FIG. 9 is a top perspective view of the female Luer fitting disinfection device shown in FIG. 4 with the sealing web removed; and

FIG. 10 is a flowchart illustrating a method according to an embodiment of the disclosure.

To facilitate understanding, identical reference numerals have been used, where possible, to designate identical elements that are common to the figures. The figures are not drawn to scale.

### DETAILED DESCRIPTION

Before describing several exemplary embodiments of the disclosure, it is to be understood that the disclosure is not limited to the details of construction or process steps set forth in the following description. The disclosure is capable of other embodiments and of being practiced or being carried out in various ways.

The matters exemplified in this description are provided to assist in understanding exemplary embodiments of the disclosure. Before describing several exemplary embodiments of the disclosure, it is to be understood that the disclosure is not limited to the details of construction or process steps set forth in the following description. The disclosure is capable of other embodiments and of being practiced or being conducted in many ways.

With respect to terms used in this disclosure, the following definitions are provided. As used herein, the use of “a,” “an,” and “the” includes the singular and plural.

Reference to “medical device” refers to any device intended to be used for medical purposes, for example, devices including Luer connector such as male and female Luer connectors. Particular embodiments pertain to a disinfection device configured to clean female Luer connectors. Embodiments of the disclosure pertain to a female Luer fitting disinfection device configured to be connected to and to disinfect a female Luer fitting. Embodiments of the disclosure advantageously provide effective disinfection of a female Luer fitting while preventing fluid path ingress of the disinfectant.

As used herein, the term “catheter related bloodstream infection” or “CRBSI” refers to any infection resulting from the presence of a catheter or IV line. As used herein, the term “Luer connector” or “Luer fitting” refers to a connection type that is the standard way of attaching syringes, catheters, hubber needles, IV tubes, etc. to each other. The Luer connector or Luer fitting consists of male and female interlocking tubes, slightly tapered to hold together better with even just a simple pressure/twist fit. Luer connectors or Luer fittings can optionally include an additional outer rim of threading, allowing them to be more secure. The Luer connector/fitting male end can interlock and connect to the female end located on the vascular access device (VAD). A Luer connector or fitting comprises a distal end, a proximal end, an irregularly shaped outer wall, a profiled center passageway for fluid communication from the chamber of the barrel of a syringe to the hub of a VAD. A Luer connector or fitting also has a distal end channel that releasably attaches the Luer connector or fitting to the hub of a VAD, and a proximal end channel that releasably attaches the Luer connector to the barrel of a syringe. As used herein, “female Luer fitting” refers to a connection collar that is the standard way of attaching syringes, catheters, hubbed needles, IV tubes, etc. to each other. Female Luer connectors or fittings include, but are not limited to, stopcocks and catheter hubs (e.g., peripherally inserted venous catheters (PIVCs), peripherally inserted central catheters (PICCs), central venous catheters (CVCs)).

A PIVC is a thin, flexible tube used for frequent intravenous medication administration. A PICC is a thin, flexible tube that is inserted into a vein in the upper arm and guided (threaded) into a large vein above the right side of the heart called the superior vena cava. A PICC is used to give intravenous fluids, blood transfusions, chemotherapy, and other drugs. A CVC is a thin, flexible tube that is inserted into a vein, usually below the right collarbone, and guided (threaded) into a large vein above the right side of the heart called the superior vena cava.

Embodiments of the female Luer fitting disinfection device of the present disclosure comprise a container comprising a bottom surface, a side wall, and an open end, a porous foam insert disposed within the container, the porous foam insert comprising a central region, a plug positioned within the central region, the plug sized and shaped to fit within a lumen of the female Luer fitting, and a liquid disinfectant in the container and absorbed within the porous foam insert, a sealing web covering the open end of the container.

In one or more embodiments of the disclosure, the porous foam insert comprises an outer peripheral portion that is precut to form a plurality of outer peripheral segments. The porous foam insert further comprises an inner peripheral portion that forms an annular ledge that is further from the open end than the outer peripheral portion. The porous foam insert is compressible upon application of a distally directed force with the female Luer fitting. The annular ledge provides a disinfecting surface to disinfect a distal surface of the female Luer fitting and the plurality of outer peripheral segments are configured to contact an outer peripheral surface of the female Luer fitting, for example, lugs or threads.

In one or more embodiments of the disclosure, the plug is removably attached to the container via a snap joint on a protrusion extending from a bottom surface of the container via a friction fitting. The container and foam insert are rotatable relative to the plug. In some embodiments, the plug is inserted and forms an interference in the female Luer fitting. Upon insertion, the disinfectant in contact with the plug disinfects the lumen of the female Luer fitting. In exemplary implementation of embodiments of the disclosure, the plug prevents ingress of the disinfectant absorbed within the porous foam insert that is greater than a predetermined safety threshold limit.

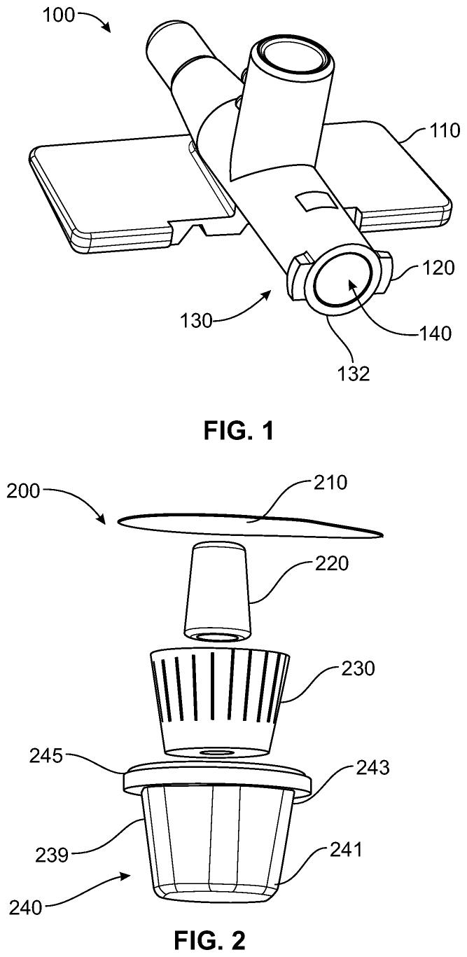

Referring now to FIG. 1, a catheter device 100 is shown comprising a female Luer fitting 130 having lumen 140 configured to allow fluid to flow therethrough. The female Luer fitting comprises a distal end 132 and a pair of lugs 120 on the outer peripheral edge of the distal end 132 of the Luer fitting 130. In the catheter device 100 shown, the catheter device 100 comprises a winged portion 110 to facilitate attachment to a patient. However, the catheter device 100 shown is just one example of a variety of medical devices having Luer fittings that may be cleaned by the disinfection devices disclosed herein. For example, the cleaning devices disclosed herein are configured to clean any medical device with a Luer fitting 130, particularly a female Luer fitting. Thus, the cleaning devices described are configured to clean medical devices with or without the winged portion 110 or lugs 120 as shown. For example, the lugs 120 could be replaced by threads that are configured to engage a locking collar (not shown).

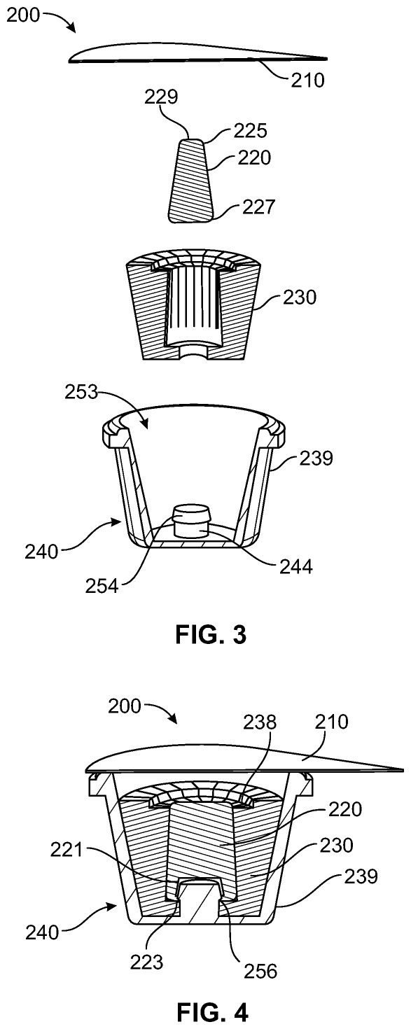

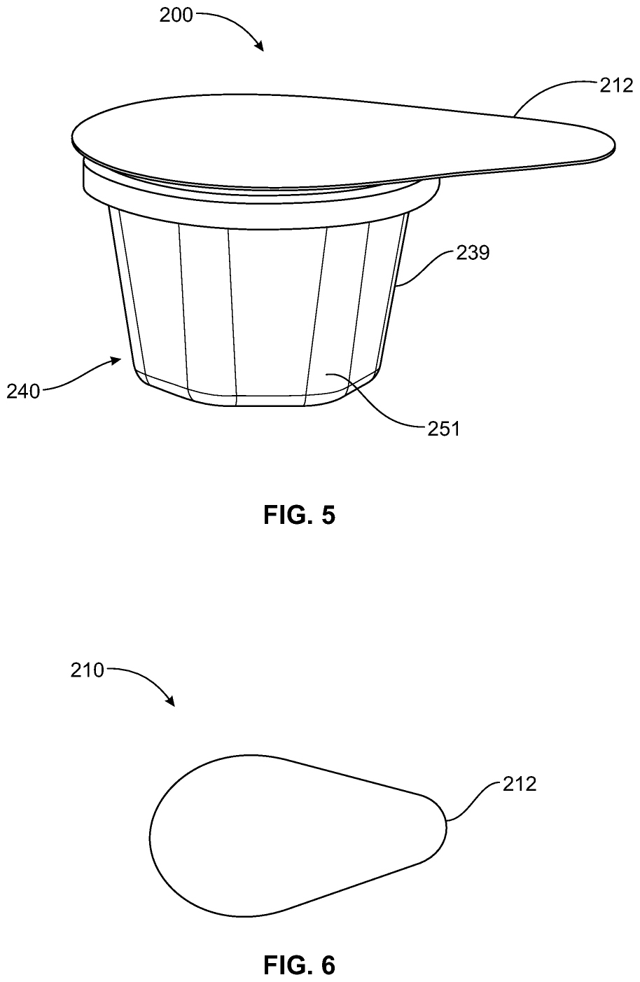

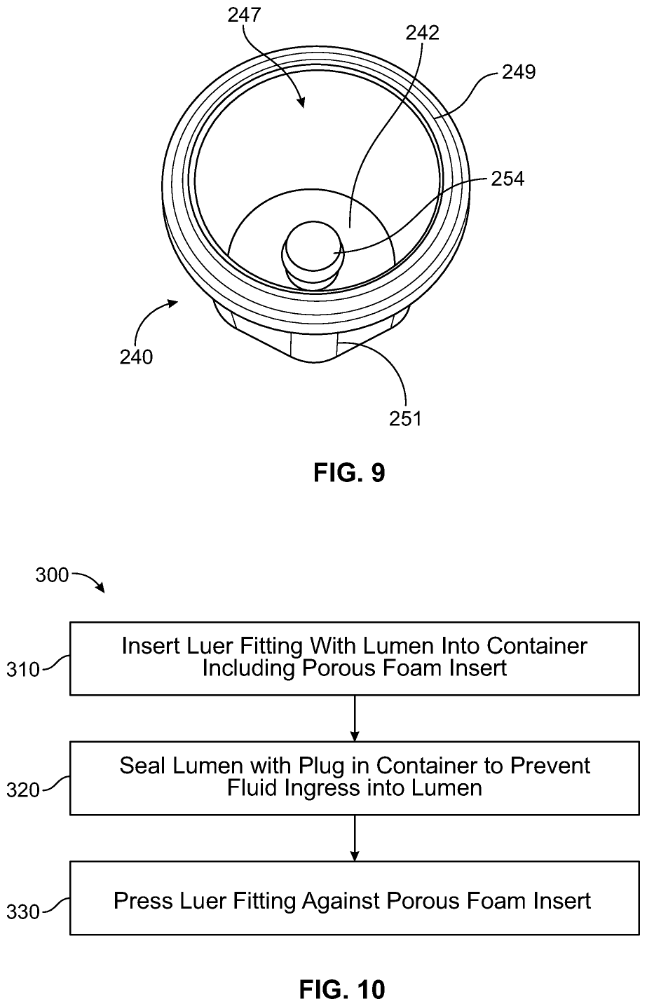

Referring now to FIGS. 2-9, and in particular FIGS. 2 and 9, a first aspect of the disclosure pertains to a disinfection device 200, particularly for disinfecting a female Luer fitting. The disinfection device 200 comprises a container 240, a porous foam insert 230, a plug 220, and a sealing web 210 sized and configured to cover and seal the contents of the container. In one or more embodiments, the container 240 is cup-shaped, having a tapered shape wherein the container 240 has a diameter at an upper portion 243 that is greater than a diameter of the container 240 at a lower portion 241. In the embodiment shown, the container 240 comprises a peripheral rim 245 having an upper peripheral surface 249 configured to form a fluid tight seal with the sealing web 210. The container 240 according to one or more embodiments is in the form of a plastic cap which houses the various components within the container as described further herein. In one or more embodiments, the container 240 comprises a recyclable material. As used herein, “recyclable” refers to materials that can be converted to new materials and objects and not disposed of in a landfill. In some embodiments, the container comprises a thermoplastic polymer or a thermoplastic elastomer. Non-limiting examples of materials include polypropylene and polyethylene, and the container material in some embodiments may have a hardness in the range but not limited to 25-100 Shore A, for example, 45-80 Shore A. The sealing web 210 according to one or more embodiments is bonded to the upper peripheral surface 249 using a heat seal, a pressure seal or a pressure-heat seal, which maintains the container closure integrity. In other embodiments, the sealing web is bonded to the container with pressure sensitive adhesive, thermal melting or induction sealing.

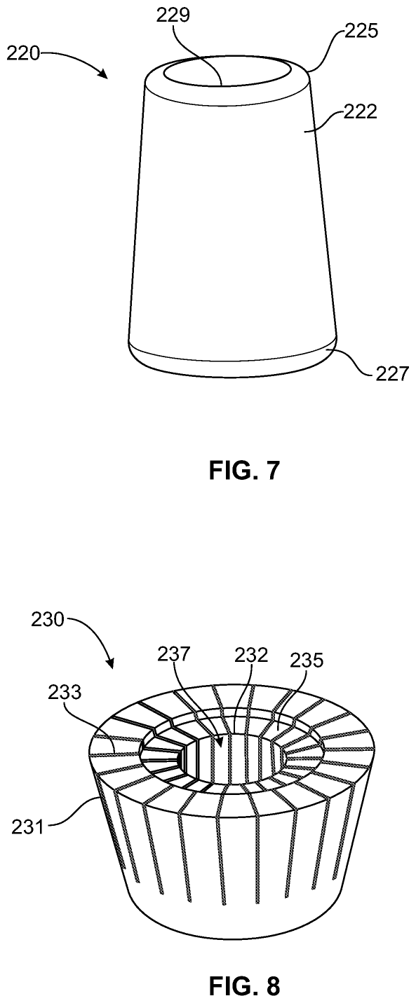

Referring to FIG. 9, the container 240 comprises a cavity 247 defined by a bottom surface 242 and a side wall 239 extending from the bottom surface 242, the side wall 239 terminating with an open end 253. The container 240 further comprises a protrusion 244 in the form of a post having an enlarged end 254 providing an undercut 256 to facilitate attachment of the plug 220 to the container 240. In some embodiments, the container 240 comprises a plurality of external ribs 251 on the side wall 239. Referring to FIG. 7, the plug 220 comprises an upper end 225 defining a tip 229 and a lower end 227 including a receiving pocket 221. The receiving pocket 221 defines a cavity including an inwardly protruding peripheral lip 223 that is configured to permit the plug 220 to snap fit with the protrusion 244. The plug 220 generally comprises a flexible, non-porous material such as an elastomer or a soft plastic. The peripheral lip 223 engage the undercut 256 to securely hold the plug 220 to the container. The plug 220 and the tip 229 are dimensioned to provide a fluid tight seal with a lumen of a female Luer fitting, for example, the lumen 140 shown in FIG. 1. Thus, the plug 220 is sized and shaped to fit within the lumen 140 of the female Luer fitting 130.

A liquid disinfectant is contained within the container and absorbed within the porous foam insert 230. The external ribs 251 are configured to provide a gripping surface for a practitioner using the Luer fitting disinfection device 200 when the practitioner twisting the Luer fitting disinfection device 200 during a cleaning operation. The container 240 and the porous foam insert 230 are rotatable with respect to the plug 220.

FIG. 6 shows the sealing web 210 comprising a cover portion sized and configured to cover the container 240 and a non-adhesive pull tab 212 configured to be gripped by a practitioner to remove the sealing web 210 from the container to expose the contents of the container 240. The sealing web in some embodiments is made from a recyclable material, which in some embodiments comprises plastic and/or a metal foil.

Referring now to FIG. 8, the porous foam insert 230 comprises a central region 237 defined and surrounded by an outer peripheral portion 231 and an inner peripheral portion 232. The outer peripheral portion 231 according to one or more embodiments comprises a plurality of outer peripheral disinfection segments 233, and the inner peripheral portion 232 comprises a plurality of inner peripheral segments 235. The inner peripheral portion 232 forms an annular ledge 238 that is further from the open end 253 than the outer peripheral portion 231. The plug 220 is positioned within the central region 237 and is secured to the protrusion 244 as described above. The outer peripheral disinfection segments 233 are configured to clean the outer periphery of the Luer fitting, including the lugs 120 (or threads if present), while the annular ledge cleans the distal end 132 of the Luer fitting. The plurality of outer peripheral disinfection segments 233 and the plurality of inner peripheral segments 235 can be precut or cut before insertion of the porous foam insert 230 is assembled to the container. The plurality of outer peripheral disinfection segments 233 in some embodiments are configured to move individually and function similar to bristles of a scrub brush to clean the outer periphery of the Luer fitting 130. Likewise, the inner peripheral segments 235 in some embodiments are configured to move individually and function as bristles of a scrub brush to clean the distal end 132 of the Luer fitting 130.

The porous foam insert 230 is compressible upon application of a distally directed force by the Luer fitting 130, namely, pressing the Luer fitting 130 into the container against the porous foam insert 230. The plug 220, which in some embodiments is removably attached to the container, is snap fit and/or interference fit with the protrusion 244. By making the plug 220 removable, a single container 240 can be used with different sized plugs to allow a wide variety of different sized Luer fittings to be cleaned. This provides for greater ease of manufacturing the disinfection device 200, as a single container may be made to fit with various sized and/or shaped plugs. The container and the porous foam insert 230 are rotatable with respect to the plug 220, which is fitted within the Luer fitting 130 to prevent fluid ingress into the lumen 140. A small amount of liquid disinfectant may be in contact with the plug 220 by surface tension or adhering to the plug 220, and the plug 220 and liquid disinfectant are configured to disinfect the lumen. In some embodiments, the plug 220 configured to prevent ingress of the liquid disinfectant absorbed within the porous foam insert 230 that is greater than a predetermined safety threshold limit. In some embodiments, the plug 220 has a frustoconical cross section and a diameter sized to form a tight seal with the Luer fitting 130. Because the plug 220 is flexible a tight seal between the plug 220 and the Luer fitting 130. In one or more embodiments, the plug 220 is sized to fit various Luer fitting opening sizes. In one or more embodiments, the safety threshold will depend on the frequency of disinfecting Luer fitting. As a non-limiting example, isopropyl alcohol blood toxicity levels are in a range of 25 and 520 mg/dl, and this translates to a limit of 1130-7700 μg as an amount per injection. According to one or more embodiments, the liquid disinfectant is selected from the group consisting of isopropyl alcohol, chlorhexidine gluconate in isopropyl alcohol, and chlorhexidine acetate in isopropyl alcohol.

The sealing web 210 is removably attached to the container 240. The sealing web 210 comprises at least one or both aluminum and a multi-layer polymer. The porous foam insert 230 in one or more embodiments comprises polyurethane foam.

Referring now to FIG. 10, another aspect of the disclosure pertains to a method 300 of disinfecting a female Luer fitting 130 of the type shown in FIG. 1. In an embodiment, the method includes at operation 310 inserting the female Luer fitting having a lumen into a container including a porous foam insert. At operation 320, the method includes sealing the lumen with a plug located in the container to prevent fluid ingress into the lumen. At operation 330, the method includes pressing the female Luer fitting against the porous foam insert. At operation 330, the method according to some embodiments further includes and rotating the container with respect to the female Luer fitting.

The method 300 according to one or more embodiments is performed using the disinfection device 200 described herein. For example, the disinfection device 200 includes the foam insert 230 comprising the annular ledge 238 surrounded by a plurality of outer peripheral disinfection segments 233. The method may further comprise pressing the female Luer fitting against the annular ledge 238. The annular ledge 238 disinfects a distal end 132 of the female Luer fitting 130. The plurality of outer peripheral disinfection segments 233 disinfect an outer peripheral surface of the of the female Luer fitting. During disinfection, in some embodiments of the method, the plug 220 remains stationary with respect to the female Luer fitting during rotation of the container while the container and the foam rotate with respect to the female Luer fitting.

Another aspect of the disclosure pertains to a method of manufacturing the disinfection device 200 described here. In one embodiment, a disinfection device is manufactured by providing the separate parts, namely, the container 240, the porous foam insert 230, the plug 220 and the sealing web 210. The porous foam insert 230 is placed in the container, and the plug 220 is press-fit onto the protrusion 244. The porous foam insert 230 either is pre-soaked with a disinfectant or the disinfectant can be soaked into the porous foam insert after it has been placed in the container. After the porous foam insert 230 and the plug have been placed in the container 240, the sealing web 210 is then affixed to the container 240. The sealing web can be affixed to the container as described herein, for example, by heat sealing, pressure sealing, a combination of pressure sealing and heating sealing or by using an adhesive.

The disinfection device 200 described herein according to one or more embodiments provides the plug 220 that provides a reliable seal to minimize fluid ingress into the lumen 140. Existing devices may not adequately provide this function due to ineffective sealing and plugging of a lumen. Many existing devices are also not specifically configured to scrub a female Luer fitting and lack the ledge feature with the inner peripheral segments and the outer peripheral segments and are thus unable to provide adequate disinfection of both the distal end of the Luer fitting and the lugs of the Luer fitting. According to one or more embodiments, the precut segments provide an excellent scrubbing surface for the entire female Luer fitting, including the distal end and the outer periphery include lugs or threads. The disinfection device according to one or more embodiments works on the principle of active disinfection of the female Luer fitting, combining the effects of mechanical scrubbing to physically dislodge microbes, and chemical action due to disinfectant. In addition, the removable plug allows the disinfection device to be configured to clean a wide variety of differently sized and shaped Luer fittings.

Reference throughout this specification to “one embodiment,” “certain embodiments,” “one or more embodiments” or “an embodiment” means that a particular feature, structure, material, or characteristic described in connection with the embodiment is included in at least one embodiment of the disclosure. Thus, the appearances of the phrases such as “in one or more embodiments,” “in certain embodiments,” “in one embodiment” or “in an embodiment” in various places throughout this specification are not necessarily referring to the same embodiment of the disclosure. Furthermore, the particular features, structures, materials, or characteristics may be combined in any suitable manner in one or more embodiments.

Although the disclosure herein has been described with reference to particular embodiments, it is to be understood that these embodiments are merely illustrative of the principles and applications of the present disclosure. It will be apparent to those skilled in the art that various modifications and variations can be made to the method and apparatus of the present disclosure without departing from the spirit and scope of the disclosure. Thus, it is intended that the present disclosure include modifications and variations that are within the scope of the appended claims and their equivalents.

What is claimed is:

1. A female Luer fitting disinfection device configured to disinfect a female Luer fitting of a medical device, the female Luer fitting disinfection device comprising:
a container comprising a bottom surface, a side wall, and an open end;
a porous foam insert disposed within the container, the porous foam insert comprising a central region;
a plug positioned within the central region, the plug sized and shaped to fit within a lumen of the female Luer fitting;
a liquid disinfectant in the container and absorbed within the porous foam insert; and
a sealing web covering the open end of the container.

2. The female Luer fitting disinfection device of claim 1, wherein the porous foam insert comprises an outer peripheral portion that is precut to form a plurality of outer peripheral segments.

3. The female Luer fitting disinfection device of claim 2, the porous foam insert further comprising an inner peripheral portion that forms an annular ledge that is further from the open end than the outer peripheral portion.

4. The female Luer fitting disinfection device of claim 3, wherein the porous foam insert is compressible upon application of a distally directed force with the female Luer fitting.

5. The female Luer fitting disinfection device of claim 4, wherein the annular ledge provides a disinfecting surface to disinfect a distal surface of the female Luer fitting and the plurality of outer peripheral segments are configured to contact an outer peripheral surface of the female Luer fitting.

6. The female Luer fitting disinfection device of claim 1, wherein the plug is removably attached to the container.

7. The female Luer fitting disinfection device of claim 6, wherein is the plug removably attached to the container via a snap joint on a protrusion extending from the bottom surface of the container.

8. The female Luer fitting disinfection device of claim 6, wherein the plug is removably attached to a protrusion extending from the bottom surface of the container via a friction fitting.

9. The female Luer fitting disinfection device of claim 1, wherein the plug is interference fit in the female Luer fitting.

10. The female Luer fitting disinfection device of claim 1, wherein the container and foam insert are rotatable relative to the plug.

11. The female Luer fitting disinfection device of claim 1, wherein the liquid disinfectant is in contact with the plug and disinfects the lumen of the female Luer fitting.

12. The female Luer fitting disinfection device of claim 11, wherein the plug prevents ingress of the liquid disinfectant absorbed within the porous foam insert that is greater than a predetermined safety threshold limit.

13. The female Luer fitting disinfection device of claim 1, wherein the liquid disinfectant is selected from the group consisting of isopropyl alcohol, chlorhexidine gluconate in isopropyl alcohol, and chlorhexidine acetate in isopropyl alcohol.

14. The female Luer fitting disinfection device of claim 1, wherein the sealing web is removable.

15. The female Luer fitting disinfection device of claim 13, wherein the sealing web comprises at least one of aluminum and a multi-layer polymer.

16. The female Luer fitting disinfection device of claim 13, wherein the sealing web is heat sealed or induction sealed to the container.

17. The female Luer fitting disinfection device of claim 1, wherein the container comprises a thermoplastic polymer.

18. The female Luer fitting disinfection device of claim 1, wherein the porous foam insert comprises polyurethane.

19. The female Luer fitting disinfection device of claim 1, wherein the plug comprises an elastomer.

20. A method of disinfecting a female Luer fitting, the method comprising:
inserting the female Luer fitting having a lumen into a container including a porous foam insert;
sealing the lumen with a plug located in the container to prevent fluid ingress into the lumen; and
pressing the female Luer fitting against the porous foam insert and rotating the container with respect to the female Luer fitting.

21. The method of claim 20, wherein the porous foam insert comprises a peripheral ledge surrounded by a plurality of disinfection segments.

22. The method of claim 21, wherein the method further comprises pressing the female Luer fitting against the peripheral ledge.

23. The method of claim 22, wherein the peripheral ledge disinfects a distal end of the female Luer fitting.

24. The method of claim 23, wherein the plurality of disinfection segments disinfect an outer peripheral surface of the of the female Luer fitting.

25. The method of claim 20, wherein the plug remains stationary during rotation of the container while the container and the porous foam insert rotates with respect to the female Luer fitting.
